(19) United States
(12) Patent Application Publication
(10) Pub. No.: US 2024/0139448 A1
(43) Pub. Date: May 2, 2024

(54) SYSTEMS AND METHODS FOR ANALYZING FIT OF A USER INTERFACE

(71) Applicant: ResMed Digital Health Inc., San Diego, CA (US)

(72) Inventors: Nathan Zersee Liu, Hornsby, NSW (AU); Albert Jack Greenwood Woffenden, Dulwich Hill, NSW (AU); Andrew Chan, Parramatta, NSW (AU); Gregory Robert Peake, Woolloomooloo, NSW (AU); Jaiden James Choy, Sydney (AU)

(21) Appl. No.: 18/494,760

(22) Filed: Oct. 25, 2023

Related U.S. Application Data
(60) Provisional application No. 63/381,287, filed on Oct. 27, 2022.

(51) Int. Cl. A61M 16/00; A61M 16/06

(52) U.S. Cl. CPC A61M 16/024; A61M 16/0622; A61M 2205/15; A61M 2205/3306; A61M 2205/3375; A61M 2205/584

(57) ABSTRACT

Methods and systems are disclosed related to determining whether there is a proper fit between a user interface and a face of a user. The methods and systems involve generating seal information associated with a seal region between a face of a user and a current user interface donned on the face of the user. The methods and systems are further related to analyzing the seal information to determine whether a leak exists in the seal region. If the leak exists, the systems and methods include analyzing the seal information to determine a location of the leak within the seal region. The systems and methods further include determining a new user interface to replace the current user interface based on the current user interface and the location of the leak.

### CROSS-REFERENCE TO RELATED APPLICATIONS

This application claims the benefit of, and priority to, U.S. Provisional Patent Application No. 63/381,287 filed on Oct. 27, 2022, which is hereby incorporated by reference herein in its entirety.

### TECHNICAL FIELD

The present disclosure relates generally to systems and methods for analyzing fit of a user interface for a user, and more particularly, to systems and methods for determining a proper user interface for a user based on the presence of leaks.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. However, some users find such systems to be uncomfortable, difficult to use, expensive, aesthetically unappealing and/or fail to perceive the benefits associated with using the system. Moreover, in some cases, users may have incorrect user interfaces for the specific user, which results in leaks of pressurized air between the face of the user and the user interface. As a result, some users will elect not to use the respiratory therapy system or discontinue use of the respiratory therapy system. The present disclosure is directed to solving these and other problems.

### SUMMARY

According to some implementations of the present disclosure, a method includes generating seal information associated with a seal region between a face of a user and a current user interface donned on the face of the user. The method also includes analyzing the seal information to determine whether a leak exists in the seal region. If the leak exists, the method also includes analyzing the seal information to determine a location of the leak within the seal region. If the leak exists, the method also includes determining a new user interface to replace the current user interface based on the current user interface and the location of the leak.

According to some implementations, the method also includes scanning, with at least one microphone, the seal region between the face of the user and the current user interface donned on the face of the user while positive airway pressure is being supplied to the user through the current user interface. The seal information is the generated by the at least one microphone during the of scanning the seal region.

According to some implementations, the method also includes the microphone being within a wearable device or a smart phone. According to these implementations, the method further includes tracing the seal region with the at least one microphone during the scanning of the seal region such that the seal information is generated as a function of a position along the seal region.

According to some implementations, the method also includes scanning, with at least one camera, the seal region between the face of the user and the current user interface donned on the face of the user. The seal information is then generated by the at least one camera during the scanning of the seal region. According to these implementations, the method further includes scanning, with the at least one camera, the face of the user prior the current user interface being donned on the face of the user to generate face information. The seal information is then generated based, at least in part, on the face information. The method can also include placing a dye on a surface of the current user interface that makes contact with the face of the user when the current user interface is donned on the face of the user. The scanning of the seal region can then include scanning the dye left on the face of the user around the seal region after removing the current user interface from being donned on the face. The seal information is then generated by the at least one camera during the scanning of the dye. The scanning of the seal region can occur after removing the current user interface from being donned on the face. In which case, the generated seal information is then based on visually detecting one or more indentations on the face of the user or on the current user interface along the seal region.

According to some implementations, the generating the seal information occurs after a period of time has elapsed from when the current user interface was donned on the face of the user so that the current user interface is fully settled on the face of the user.

According to some implementations, the current user interface includes a dye that is time-activated, moisture activated, photochromic, ultraviolet light sensitive, or a combination thereof. According to these implementations, the dye can be on a peelable layer on the current user interface.

According to some implementations, the seal information can be based on audio information generated from nasal resistance.

According to some implementations of the present disclosure, another method includes providing a current user interface connected to a respiratory therapy system. The current user interface includes a seal surface where the current user interface contacts a face of a user with the current user interface donned on the face of the user. The method also includes providing an indicator on the seal surface of the user interface. The indicator is configured to contact the face of the user when the current user interface is donned on the face of the user. The method also includes generating seal information associated with a seal region between the face of the user and the seal surface of the current user interface based on the indicator and upon the current user interface being removed from the face of the user. The method also includes analyzing the seal information to determine whether the current user interface fits properly.

According to some implementations, the method includes continuing to use the current user interface if the current user interface is determined to fit properly, and returning the current user interface for a new user interface if the current user interface is determined to not fit properly.

According to some implementations, the indicator is a contour-forming material that develops an impression of topology of the face of the user.

According to some implementations, the indicator is a dye. According to these implementations, the dye can be configured to transfer to the face of the user when the current user interface is donned on the face of the user. According to these implementations, the dye alternatively can be configured to remain on the seal surface of the current user interface upon the current user interface being removed from the face of the user. According to these implementations, the dye can be time-activated, moisture activated, photochromic, ultraviolet light sensitive, or a combination thereof. According to these implementations, the method can also include providing the dye on or within a peelable layer on the seal surface of the user interface.

According to some implementations, the dye is moisture-released from the peelable layer so as to transfer to the face of the user when the current user interface is donned on the face of the user. According to some implementations, the method includes removing the peelable layer from the seal surface for continued use of the current user interface if the current user interface is determined to fit properly.

According to some implementations of the present disclosure, a system includes a memory and a control system. The memory stores machine-readable instructions. The control system includes one or more processors configured to execute the machine-readable instructions to generate seal information associated with a seal region between a face of a user and a current user interface donned on the face of the user. The one or more processors further are configured to execute the machine-readable instructions to analyze the seal information to determine whether a leak exists in the seal region. If the leak exists, the one or more processors further are configured to execute the machine-readable instructions to analyze the seal information to determine a location of the leak within the seal region. The one or more processors further are configured to execute the machine-readable instructions to determine a new user interface to replace the current user interface based on the current user interface and the location of the leak.

According to some implementations of the present disclosure, a system includes a current user interface connected to a respiratory therapy system. The current user interface includes a seal surface where the current user interface contacts a face of a user with the current user interface donned on the face of the user. The system further includes an indicator on the seal surface of the user interface. The indicator is configured to contact the face of the user when the current user interface is donned on the face of the user. The system further includes a memory and a control system. The memory stores machine-readable instructions. The control system includes one or more processors configured to execute the machine-readable instructions to generate seal information associated with a seal region between the face of the user and the seal surface of the current user interface based on the indicator and upon the current user interface being removed from the face of the user. The one or more processors further are configured to execute the machine-readable instructions to analyze the seal information to determine whether the current user interface fits properly.

According to some implementations, a user interface is disclosed. The user interface includes a frame and headgear that position the user interface on a face of a user relative to an airway of the user, with the user interface donned on the face of the user. The user interface further includes a cushion that is supported against the face of the user by the frame and the headgear to define a seal region around the airway of the user, with the user interface donned on the face of the user. The cushion includes a seal surface where the cushion contacts the face of the user at the seal region. The user interface further includes an indicator on the seal surface. The indicator is configured to contact the face of the user when the user interface is donned on the face of the user for determining whether the user interface fits properly.

According to some implementations, the user interface includes a peelable layer on the seal surface. The indicator can be the peelable layer, can be on the peelable layer, can be in the peelable layer, or a combination thereof.

According to some implementations, the indicator is a contour-forming material that develops an impression of topology of the face of the user, and the impression of topology can be analyzed for determining whether the user interface fits properly.

According to some implementations, the indicator is a dye that makes contact with the face of the user when the user interface is donned on the face of the user. According to these implementations, the dye is configured to transfer to the face of the user when the current user interface is donned on the face of the user. The transferred dye on the face of the user can then be visually scanned for determining whether the user interface fits properly. According to these implementations, the dye can be configured to activate when in contact with the face of the user but remain on the seal surface of the current user interface upon the current user interface being removed from the face of the user. The activated portions of the dye on the seal surface can be visually scanned for determining whether the user interface fits properly. According to these implementations, the dye can be time-activated, moisture activated, photochromic, ultraviolet light sensitive, or a combination thereof. According to these implementations, the user interface can further includes a peelable layer on the seal surface. The dye can be on the peelable layer, can be in the peelable layer, or a combination thereof. According to these implementations, the dye can be moisture-released from the peelable layer so as to transfer to the face of the user when the current user interface is donned on the face of the user.

According to some implementations, a cushion of a user interface is disclosed. The cushion includes a seal surface that contacts a face of a user when the user interface is donned on the face of the user. The cushion also includes an indicator on the seal surface. The indicator is configured to contact the face of the user when the user interface is donned on the face of the user for determining whether the user interface fits properly.

According to some implementations, the cushion includes a peelable layer on the seal surface. The indicator can be the peelable layer, can be on the peelable layer, can be in the peelable layer, or a combination thereof.

According to some implementations, the indicator is a contour-forming material that develops an impression of topology of the face of the user, and the impression of topology can be analyzed for determining whether the user interface fits properly.

According to some implementations, the indicator is a dye that makes contact with the face of the user when the user interface is donned on the face of the user. According to these implementations, the dye is configured to transfer to the face of the user when the user interface is donned on the face of the user. The transferred dye on the face of the user can be visually scanned for determining whether the user interface fits properly. According to these implementations, the dye is configured to activate when in contact with the face of the user but remain on the seal surface of the user interface upon the user interface being removed from the face of the user. Activated portions of the dye on the seal surface can be visually scanned for determining whether the user interface fits properly. According to these implementations, the dye is time-activated, moisture activated, photochromic, ultraviolet light sensitive, or a combination thereof. According to these implementations, the cushion includes a peelable layer on the seal surface. The dye is on the peelable layer, is in the peelable layer, or a combination thereof. According to these implementations, the dye is moisture-released from the peelable layer so as to transfer to the face of the user when the current user interface is donned on the face of the user.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3A is a perspective view of a respiratory therapy device of the system of FIG. 1, according to some implementations of the present disclosure;

FIG. 3B is a perspective view of the respiratory therapy device of FIG. 3A illustrating an interior of a housing, according to some implementations of the present disclosure;

FIG. 4A is a perspective view of a user interface, according to some implementations of the present disclosure;

FIG. 4B is an exploded view of the user interface of FIG. 4A, according to some implementations of the present disclosure;

FIG. 4C is a perspective view of the cushion of the user interface of FIG. 4A, according to some implementations of the present disclosure;

FIG. 4D is another perspective view of the cushion of the user interface of FIG. 4A, according to some alternative implementations of the present disclosure;

FIG. 5A is a perspective view of a user interface, according to some implementations of the present disclosure;

FIG. 5B is an exploded view of the user interface of FIG. 5A, according to some implementations of the present disclosure;

FIG. 6A is a perspective view of a user interface, according to some implementations of the present disclosure;

FIG. 6B is an exploded view of the user interface of FIG. 6A, according to some implementations of the present disclosure;

FIG. 7 illustrates an exemplary timeline for a sleep session, according to some implementations of the present disclosure;

FIG. 8 illustrates an exemplary hypnogram associated with the sleep session of FIG. 7, according to some implementations of the present disclosure;

FIG. 9A is a front view of a user donning a user interface, according to some implementations of the present disclosure;

FIG. 9B is a front view of the user of FIG. 9A after removing the donned user interface; according to some implementations of the present disclosure;

FIG. 9C is a perspective view of the cushion of the user interface of FIG. 9A, according to some implementations of the present disclosure;

FIG. 10 is a front view of a user donning a user interface, according to some implementations of the present disclosure;

FIG. 11 is a process flow diagram for a method for determining whether a user interface fits properly, according to some implementations of the present disclosure; and

FIG. 12 is a process flow diagram for another method for determining whether a user interface fits properly, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

The present disclosure is directed to systems and methods that optimize a user interface for a user, such as based on mask model and size, to prevent or reduce the likelihood of an improperly fitted user interface. The systems and methods determine the fit of the user interface based on audio and/or visual scans of the user's face, either with or without the user interface donned on the user's face. Based on the audio and/or visual scans, leaks or otherwise improperly fitted user interfaces can be determined. Thereafter, another user interface can be recommended and provided to the user that attempts to correct the issue with the previous user interface, to provide the user with a better fit. The better fit is likely to promote the user to continue use of the user interface and associated therapy.

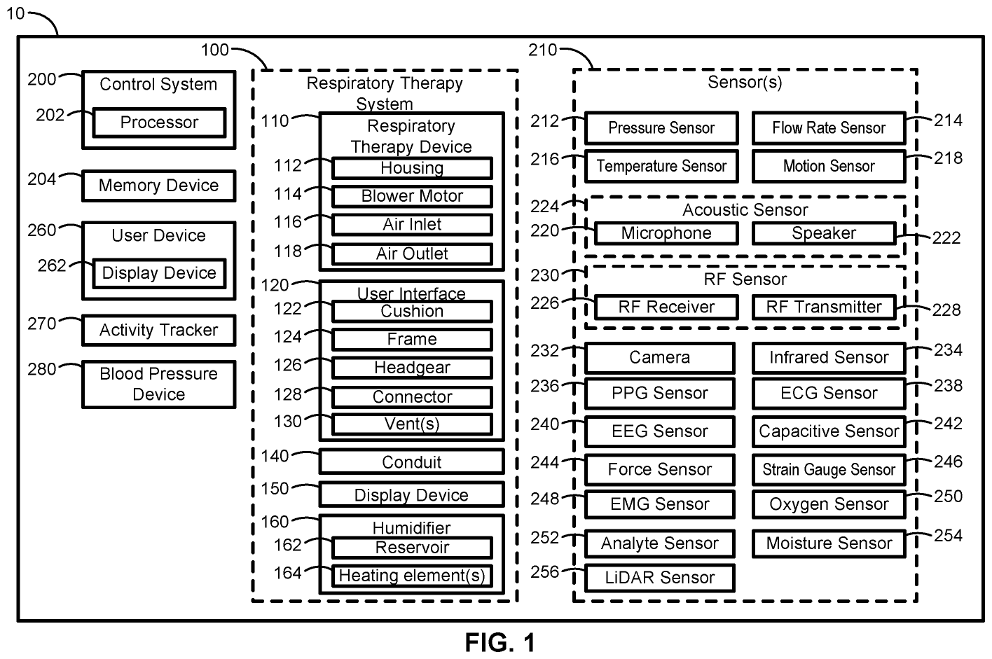

Referring to FIG. 1, a system 10, according to some implementations of the present disclosure, is illustrated. The system 10 includes a respiratory therapy system 100, a control system 200, one or more sensors 210, a user device 260, and an activity tracker 270.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

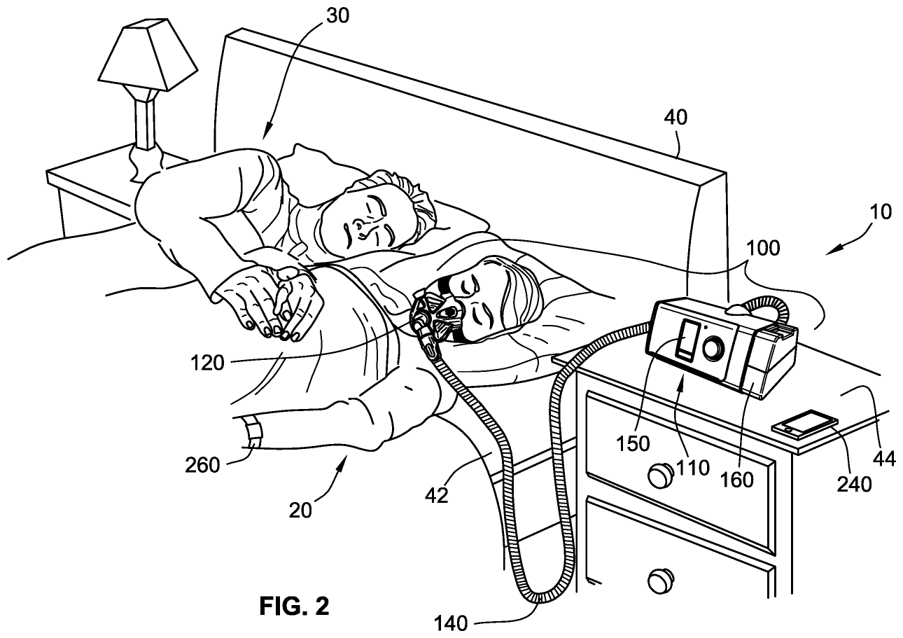

As shown in FIG. 2, the respiratory therapy system 100 can be used to treat user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

The respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure).

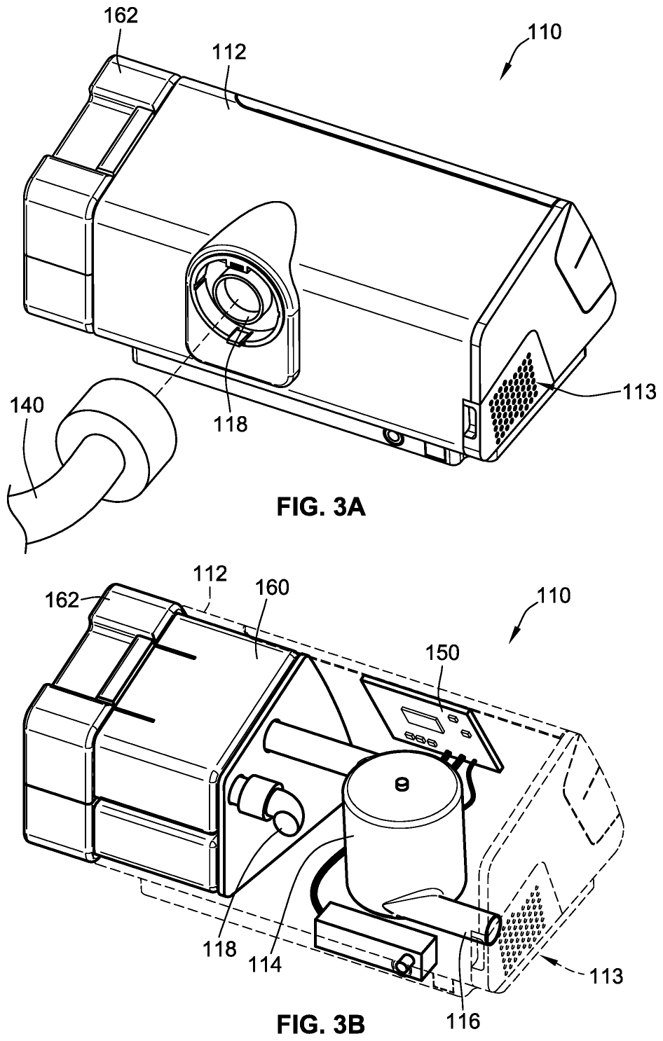

The respiratory therapy device 110 includes a housing 112, a blower motor 114, an air inlet 116, and an air outlet 118 (FIG. 1). Referring to FIGS. 3A and 3B, the blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). As shown in FIGS. 3A and 3B, the housing 112 can include a vent 113 to allow air to pass through the housing 112 to the air inlet 116. As described below, the conduit 140 is coupled to the air outlet 118 of the respiratory therapy device 110.

Referring back to FIG. 1, the user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

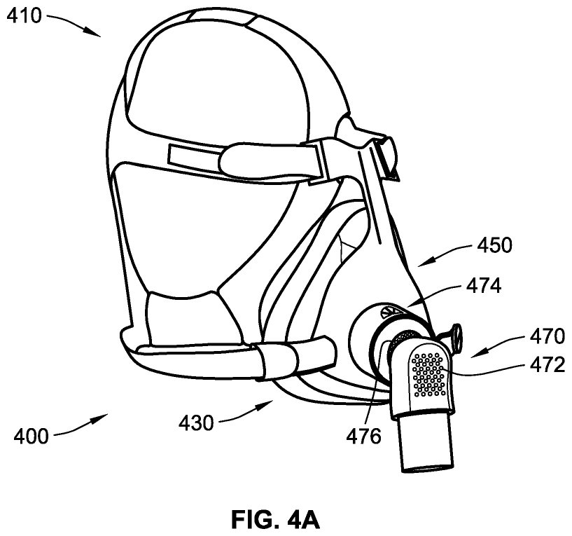

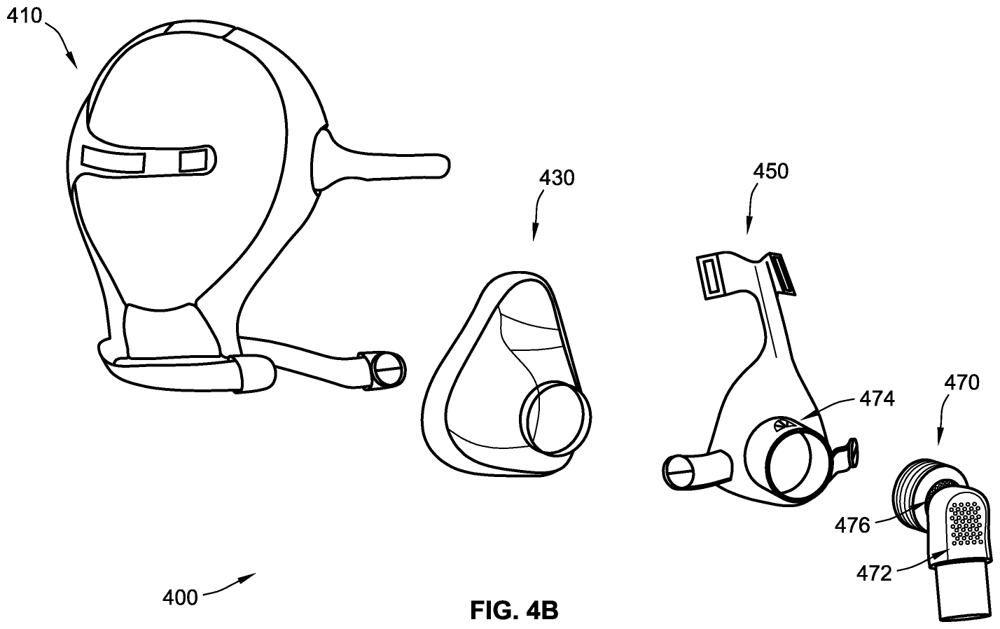

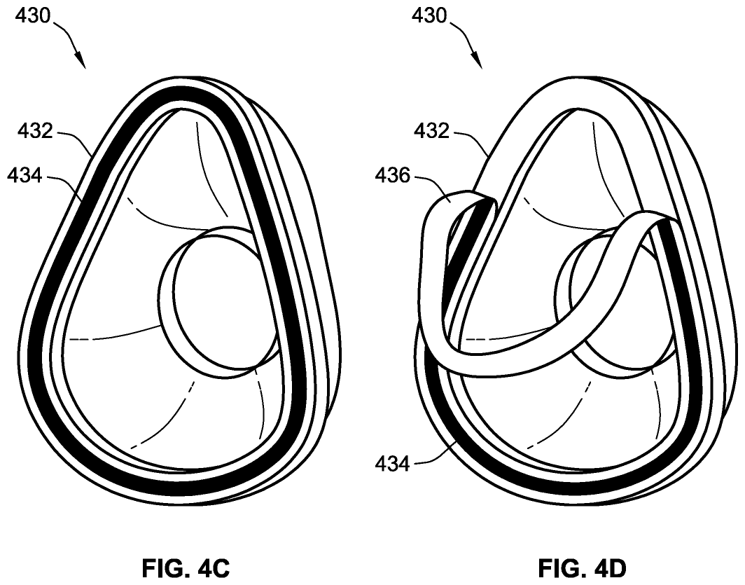

Referring to FIGS. 4A-4D, a user interface 400 that is the same as, or similar to, the user interface 120 (FIG. 1), according to some implementations of the present disclosure is illustrated. The user interface 400 generally includes a cushion 430 and a frame 450 that define a volume of space around the mouth and/or nose of the user. When in use, the volume of space receives pressurized air for passage into the user's airways. In some implementations, the cushion 430 and frame 450 of the user interface 400 form a unitary component of the user interface. The user interface 400 can also include a headgear 410, which generally includes a strap assembly and optionally a connector 470. The headgear 410 is configured to be positioned generally about at least a portion of a user's head when the user wears the user interface 400. The headgear 410 can be coupled to the frame 450 and positioned on the user's head such that the user's head is positioned between the headgear 410 and the frame 450. The cushion 430 is positioned between the user's face and the frame 450 to form a seal on the user's face. The optional connector 470 is configured to couple to the frame 450 and/or cushion 430 at one end and to a conduit of a respiratory therapy device (not shown). The pressurized air can flow directly from the conduit of the respiratory therapy system into the volume of space defined by the cushion 430 (or cushion 430 and frame 450) of the user interface 400 through the connector 470). From the user interface 400, the pressurized air reaches the user's airway through the user's mouth, nose, or both. Alternatively, where the user interface 400 does not include the connector 470, the conduit of the respiratory therapy system can connect directly to the cushion 430 and/or the frame 450.

In some implementations, the connector 470 may include one or more vents 472 (e.g., a plurality of vents) located on the main body of the connector 470 itself and/or one or a plurality of vents 476 (“diffuser vents”) in proximity to the frame 450, for permitting the escape of carbon dioxide (CO₂) and other gases exhaled by the user. In some implementations, one or a plurality of vents, such as vents 472 and/or 476 may be located in the user interface 400, such as in frame 450, and/or in the conduit 140. In some implementations, the frame 450 includes at least one anti-asphyxia valve (AAV) 474, which allows CO₂ and other gases exhaled by the user to escape in the event that the vents (e.g., the vents 472 or 476) fail when the respiratory therapy device is active. In general, AAVs (e.g., the AAV 474) are present for full face masks (e.g., as a safety feature); however, the diffuser vents and vents located on the mask or connector (usually an array of orifices in the mask material itself or a mesh made of some sort of fabric, in many cases replaceable) are not necessarily both present (e.g., some masks might have only the diffuser vents such as the plurality of vents 476, other masks might have only the plurality of vents 472 on the connector itself).

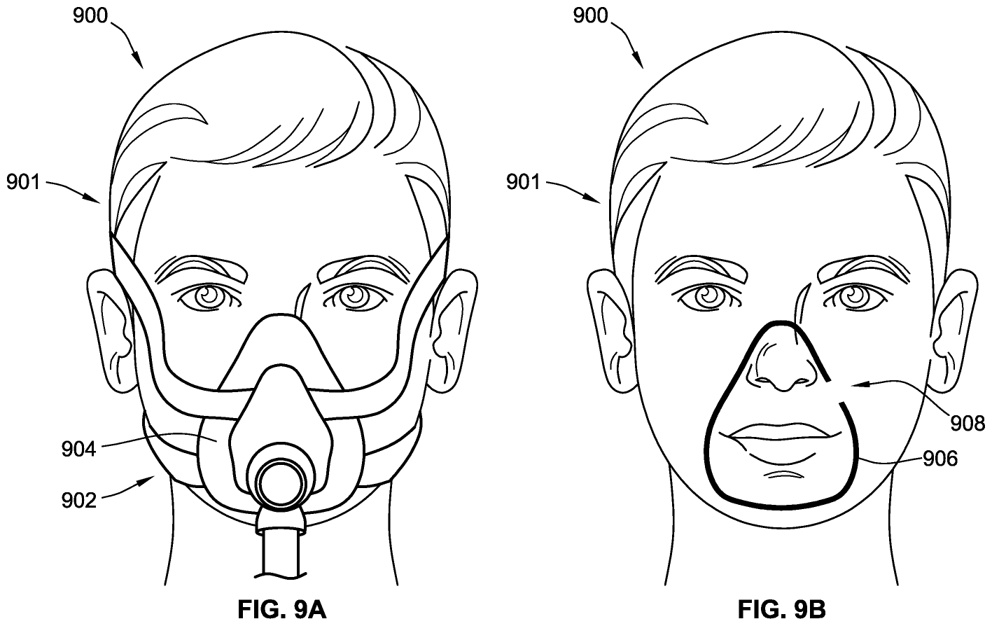

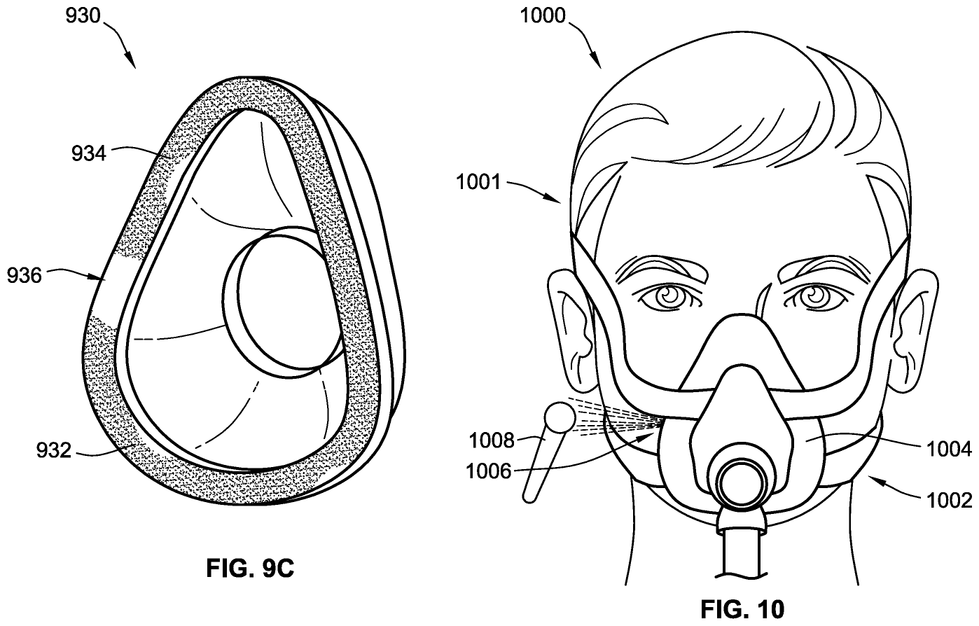

Referring to FIG. 4C, the cushion 430 is shown in greater detail, according to some implementations of the present disclosure. Specifically, the cushion 430 includes a seal surface 432. The seal surface 432 is generally the portion of the cushion 430 that contacts the face of the user (e.g., user 120 of FIG. 2). Thus, the seal surface 432 in combination with the corresponding portion of the face of the user that contacts the seal surface 432 generally constitutes the seal region, which is discussed further below with respect to FIGS. 9A-9C.

The seal surface 432 has thereon or therein an indicator 434. The indicator 434 is configured to contact the face of the user when the user interface 400 is donned on the face of the user. The indicator 434 is used for determining whether the user interface 400 fits properly on the face of the user.

According to some implementations, the indicator 434 can be in the form of a dye that makes contact with the face of the user when the user interface is donned on the face of the user. The dye (indicator 434) can be configured to transfer to the face of the user when the user interface 400 is donned on the face of the user. As discussed further below with respect to FIGS. 9A and 9B, the transferred dye (indicator 434) on the face of the user can be visually scanned for determining whether the user interface 400 fits properly.

Alternatively, the dye (indicator 434) can be configured to activate when in contact with the face of the user but remain on the seal surface 434 of the cushion 430 after the user interface 400 is removed from the face of the user. Thereafter, activated portions of the dye (indicator 434) on the seal surface 4332 can be visually scanned for determining whether the user interface 400 fits properly, as discussed further below with respect to FIG. 9C.

In both cases, where the dye (indicator 434) is configured to transfer to the face or remain on the seal surface 432, the dye (indicator 434) can be time-activated, moisture activated or released, photochromic, ultraviolet light sensitive, invisible to the naked eye, or a combination thereof.

As an alternative, or in addition, the indicator 434 can be the material that forms at least the part of the cushion 430 at the seal surface 432. For example, the cushion 430 at the seal surface 432 can be formed of a moisture activated material or a heat activated material or both. The moisture activated material or the heat activated material as the indicator 434 can be activated when in contact with the face of the user, such as by changing in color or changing in opacity or by changing in some other visual or otherwise detectable quality. Thereafter, the indicator 434 can be used to check the seal between the cushion 434 and the face of the user, such as by determining whether there is a uniform color change in the indicator 434 responsive to the heat from the skin of the user in contact with the indicator. Discontinuities in the change of the quality of the indicator, such as discontinuities in color and or opacity, indicate an improper fit. However, according to some implementations, the presence of discontinuities can indicate an improper fit. Alternatively, the presence and severity of discontinuities can indicate an improper fit. For example, the color change may not be uniform, such that there is a discontinuity in the color change. However, the severity of the color change further can indicate an improper fit. For example, a color change above a threshold, or a color change within a threshold amount of the remaining color change, can indicate a properly fitted user interface. Alternatively, a color change below a threshold, or outside of a threshold amount of the remaining color change, can indicate an improperly fitted user interface.

As an alternative, or in addition, the indicator 434 can instead be a contour-forming material that develops an impression of topology of the face of the user while the user interface 400 is donned on the face. Once removed, the contour-forming material (indicator 434) retains the impression of topology, which can then be visually scanned, such as by using a camera of a smart device. The visual scan can then be analyzed for determining whether the user interface fits properly. Such a determination can be based on, for example, whether the thickness of the contour-forming material (indicator 434) has been changed over the entire perimeter of the contour-forming material (indicator 434), which indicates that the contour-forming material (indicator 434) made contact with the face along its entire perimeter. Such a determination also can be based on, for example, whether compression of the contour-forming material (indicator 434) exceeded a threshold such that the contour-forming material (indicator 434) could not be compressed any further. This may indicate that the contact between the cushion 430 and the face of the user is too severe, which may lead to discomfort of the user overtime, failure of the cushion 430 over time, failure of the headgear of the 410 of the user interface 400, and the like.

Referring to FIG. 4D, the cushion 430 can include a peelable layer 436, which is shown in a partially peeled state. The peelable layer 436 initially can be affixed to the seal surface 432. Once used, the peelable layer 436 can be removed from the seal surface 432, as further described below. Alternatively, the peelable layer 436, when on the cushion 430, can be considered the seal surface because the peelable layer 436 is the element on the cushion 430 that makes a seal with the face of the user.

The dye (indicator 434) discussed above can be on the peelable layer 436, in the peelable layer 436, or a combination thereof. Thus, once a determination is made as to whether the user interface 400 fits properly, the peelable layer 436 with the dye (indicator 434) can be removed so that the cushion 430 does not continue to transfer dye to surfaces that it touches.

According to some implementations, the peelable layer 436 can be the indicator 434. For example, the peelable layer 436 can be a contour-forming material. Once the peelable layer 436 is used as the contour-forming material as the indicator 434 and the fit of the user interface 400 is determined, the peelable layer 436 can be removed, specifically if the user is to maintain using the user interface.

Although described above generally as being on the cushion 430, according to some implementations, the indicator 434 can be on any surface of the user interface 400 that can be directly or indirectly related to the fit of the user interface 400 on the user. For example, the indicator 434 can also or solely be arranged on the head gear 410. On the head gear 410, the indicator 434 can similarly visually or otherwise indicate the fit of the user interface 400. For example, the indicator 434 on the head gear 410 can visually indicate strain within the head gear 410, either absolutely or relative to a threshold (e.g., above a set strain threshold). The indicator 434 on the head gear 410 can further indicate whether the fit of the user interface is proper or whether the user should obtain a new user interface that may provide a proper, or more proper, fit.

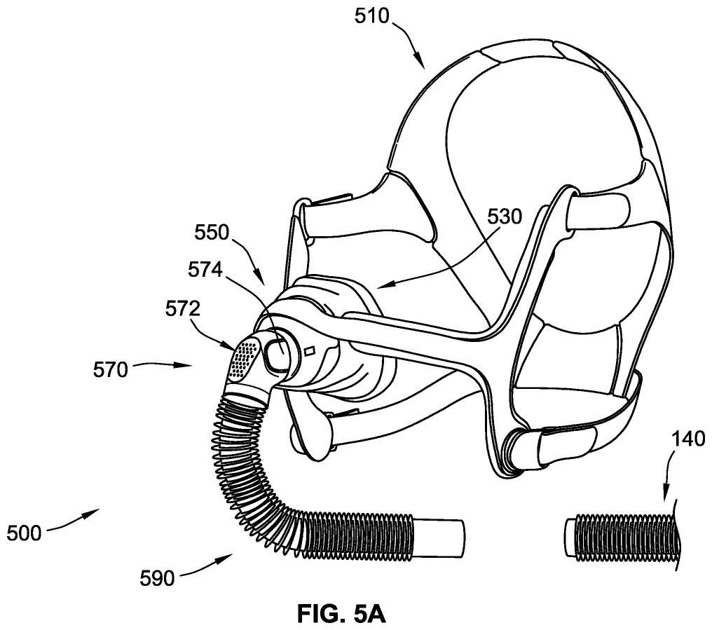

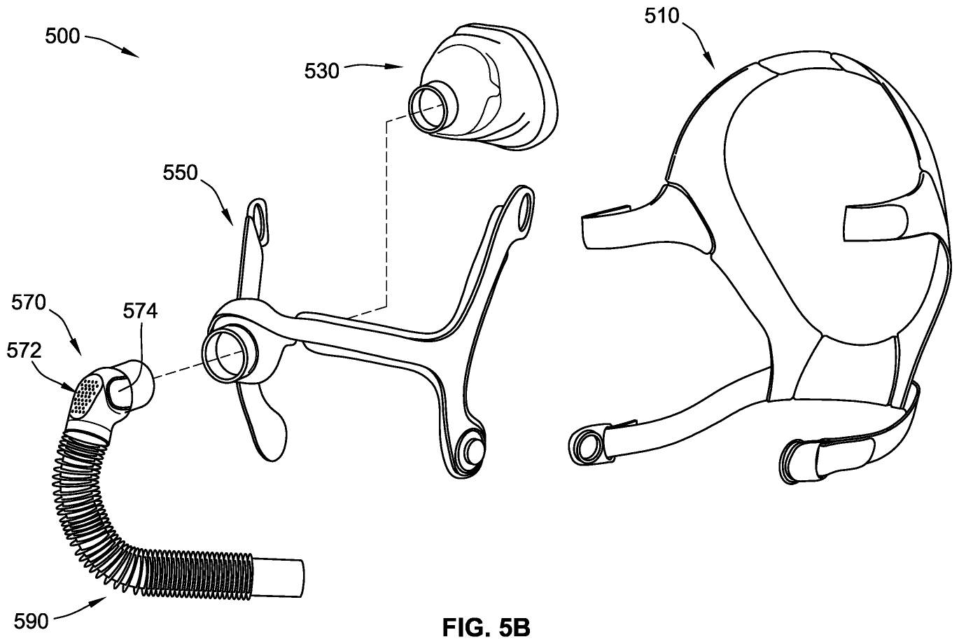

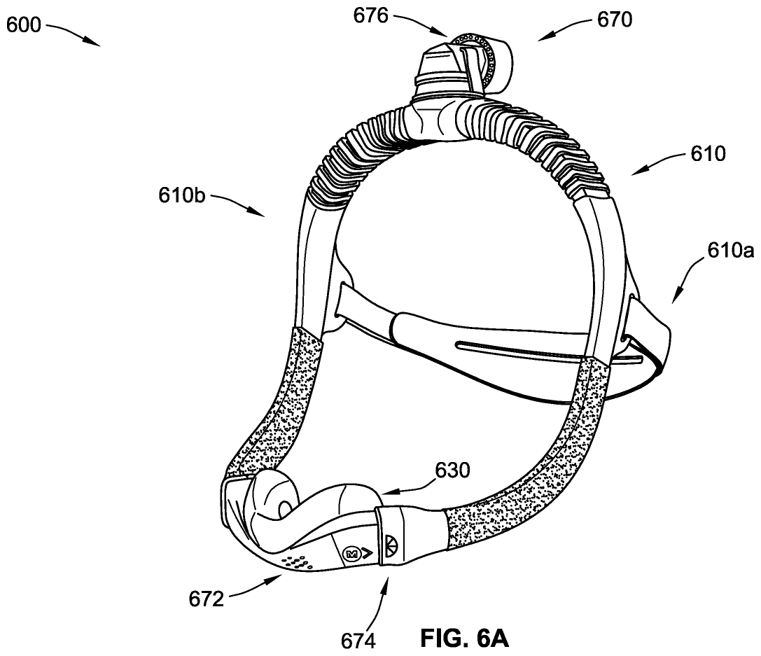

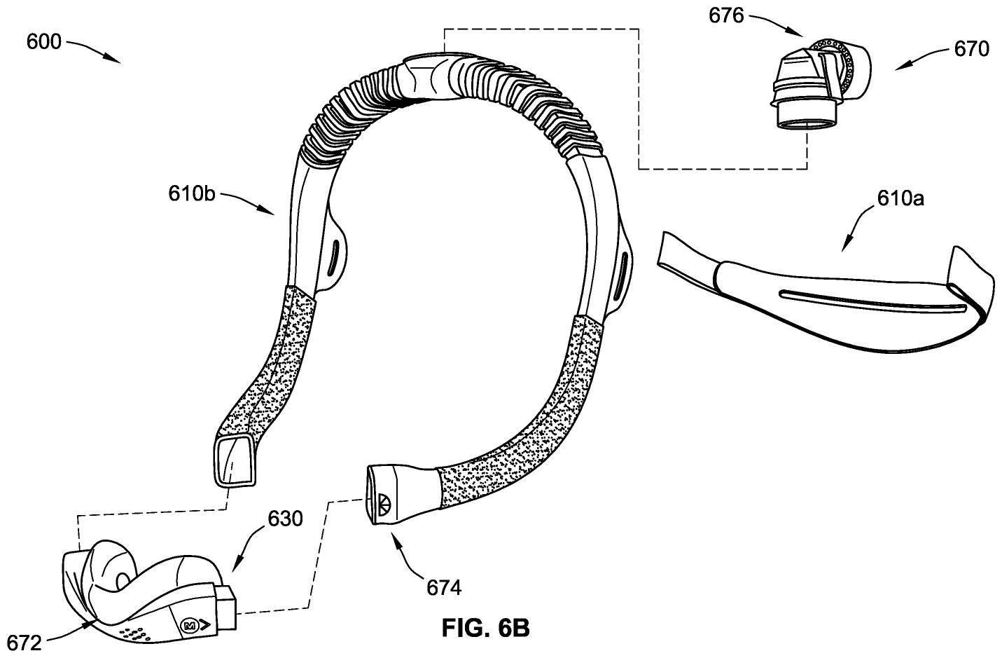

The concepts discussed above with respect to FIGS. 4A-4D and the cushion 430 and the seal surface 432 with the indicator 434 can be applied to any user interface described herein. Thus, despite the cushion 430, the seal surface 432, and the indicator 434 being discussed with respect to the user interface 400, similar seal surfaces and indicators as the seal surface 432 and the indicator 434 can be on other user interfaces, such as those described below in FIGS. 5A and 5B and in FIGS. 6A and 6B.

Referring to FIGS. 5A and 5B, a user interface 500 that the is the same, or similar to, the user interface 120 (FIG. 1) according to some implementations of the present disclosure is illustrated. The user interface 500 differs from the user interface 400 (FIGS. 4A and 4B) in that the user interface 500 is an indirect user interface, whereas the user interface 400 is a direct user interface. The interface 500 includes a headgear 510 (e.g., as a strap assembly), a cushion 530, a frame 550, a connector 570, and a user interface conduit 590 (often referred to as a minitube or a flexitube). The user interface 500 is an indirectly connected user interface because pressurized air is delivered from the conduit 140 of the respiratory therapy system to the cushion 530 and/or frame 550 through the user interface conduit 590, rather than directly from the conduit 140 of the respiratory therapy system.

In some implementations, the cushion 530 and frame 550 form a unitary component of the user interface 500. Generally, the user interface conduit 590 is more flexible than the conduit 140 of the respiratory therapy system 100 (FIG. 1) described above and/or has a diameter smaller than the diameter of the than the than the conduit 140. The user interface conduit 590 is typically shorter that conduit 140. Similar to the headgear 310 of user interface 300 (FIGS. 3A-3B), the headgear 510 of user interface 500 is configured to be positioned generally about at least a portion of a user's head when the user wears the user interface 500. The headgear 510 can be coupled to the frame 550 and positioned on the user's head such that the user's head is positioned between the headgear 510 and the frame 550. The cushion 530 is positioned between the user's face and the frame 550 to form a seal on the user's face. The connector 570 is configured to couple to the frame 550 and/or cushion 530 at one end and to the conduit 590 of the user interface 500 at the other end. In other implementations, the conduit 590 may connect directly to frame 550 and/or cushion 530. The conduit 590, at the opposite end relative to the frame 550 and cushion 530, is configured to connect to the conduit 140. The pressurized air can flow from the conduit 140 of the respiratory therapy system, through the user interface conduit 590, and the connector 570, and into a volume of space define by the cushion 530 (or cushion 530 and frame 550) of the user interface 500 against a user's face. From the volume of space, the pressurized air reaches the user's airway through the user's mouth, nose, or both.

In some implementations, the connector 570 includes a plurality of vents 572 for permitting the escape of carbon dioxide (CO₂) and other gases exhaled by the user when the respiratory therapy device is active. In such implementations, each of the plurality of vents 572 is an opening that may be angled relative to the thickness of the connector wall through which the opening is formed. The angled openings can reduce noise of the CO₂ and other gases escaping to the atmosphere. Because of the reduced noise, acoustic signal associated with the plurality of vents 572 may be more apparent to an internal microphone, as opposed to an external microphone. Thus, an internal microphone may be located within, or otherwise physically integrated with, the respiratory therapy system and in acoustic communication with the flow of air which, in operation, is generated by the flow generator of the respiratory therapy device, and passes through the conduit and to the user interface 500.

In some implementations, the connector 570 optionally includes at least one valve 574 for permitting the escape of CO₂ and other gases exhaled by the user when the respiratory therapy device is inactive. In some implementations, the valve 574 (an example of an anti-asphyxia valve) includes a silicone (or other suitable material) flap that is a failsafe component, which allows CO₂ and other gases exhaled by the user to escape in the event that the vents 572 fail when the respiratory therapy device is active. In such implementations, when the silicone flap is open, the valve opening is much greater than each vent opening, and therefore less likely to be blocked by occlusion materials.

Applying the concepts of the seal surface 432 and the indicator 434 in FIGS. 4C and 4D, the cushion 530 of the user interface 500 can include a similar seal surface and indictor as the seal surface 432 and the indicator 434, respectively, of the user interface 400 in FIGS. 4A-4D.

Referring to FIGS. 6A and 6B, a user interface 600 that is the same as, or similar to, the user interface 120 (FIG. 1) according to some implementations of the present disclosure is illustrated. The user interface 600 is similar to the user interface 500 in that it is an indirect user interface. The indirect headgear user interface 600 includes headgear 610, a cushion 630, and a connector 670. The headgear 610 includes strap 610a and a headgear conduit 610b. Similar to the user interface 400 (FIGS. 4A-4B) and user interface 500 (FIGS. 5A-5B), the headgear 610 is configured to be positioned generally about at least a portion of a user's head when the user wears the user interface 600. The headgear 610 includes a strap 610a that can be coupled to the headgear conduit 610b and positioned on the user's head such that the user's head is positioned between the strap 610a and the headgear conduit 610b. The cushion 630 is positioned between the user's face and the headgear conduit 610b to form a seal on the user's face.

The connector 670 is configured to couple to the headgear 610 at one end and a conduit of the respiratory therapy system at the other end (e.g., conduit 140). In other implementations, the connector 670 is not included and the headgear 610 can alternatively connect directly to conduit of the respiratory therapy system. The headgear conduit 610b can be configured to deliver pressurized air from the conduit of the respiratory therapy system to the cushion 630, or more specifically, to the volume of space around the mouth and/or nose of the user and enclosed by the user cushion. The headgear conduit 610b is hollow to provide a passageway for the pressurized air. Both sides of the headgear conduit 610b can be hollow to provide two passageways for the pressurized air. Alternatively, only one side of the headgear conduit 610b can be hollow to provide a single passageway. In the implementation illustrated in FIGS. 6A and 6B, headgear conduit 610b comprises two passageways which, in use, are positioned at either side of a user's head/face. Alternatively, only one passageway of the headgear conduit 610b can be hollow to provide a single passageway. The pressurized air can flow from the conduit of the respiratory therapy system, through the connector 670 and the headgear conduit 610b, and into the volume of space between the cushion 630 and the user's face. From the volume of space between the cushion 630 and the user's face, the pressurized air reaches the user's airway through the user's mouth, nose, or both.

In some implementations, the cushion 630 includes a plurality of vents 672 on the cushion 630 itself. Additionally or alternatively, in some implementations, the connector 670 includes a plurality of vents 676 (“diffuser vents”) in proximity to the headgear 610, for permitting the escape of carbon dioxide (CO₂) and other gases exhaled by the user when the respiratory therapy device is active. In some implementations, the headgear 610 may include at least one plus anti-asphyxia valve (AAV) 674 in proximity to the cushion 630, which allows CO₂ and other gases exhaled by the user to escape in the event that the vents (e.g., the vents 672 or 676) fail when the respiratory therapy device is active.

Applying the concepts of the seal surface 432 and the indicator 434 in FIGS. 4C and 4D, the cushion 630 of the user interface 600 can include a similar seal surface and indictor as the seal surface 432 and the indicator 434, respectively, of the user interface 400 in FIGS. 4A-4D

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

Referring to FIG. 3A, the conduit 140 includes a first end 142 that is coupled to the air outlet 118 of the respiratory therapy device 110. The first end 142 can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end 142 can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, as shown in FIG. 3, air flow from the air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

Referring to back to FIG. 1, the one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a LiDAR sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the infrared sensor 234, the photoplethysmogram (PPG) sensor 236, the electrocardiogram (ECG) sensor 238, the electroencephalography (EEG) sensor 240, the capacitive sensor 242, the force sensor 244, the strain gauge sensor 246, the electromyography (EMG) sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 (FIG. 2) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20 (FIG. 2), a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260.

The microphone 220 and the speaker 222 can be used as separate devices. In some implementations, the microphone 220 and the speaker 222 can be combined into an acoustic sensor 224 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30 (FIG. 2). Based at least in part on the data from the microphone 220 and/or the speaker 222, the control system 200 can determine a location of the user 20 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g. a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user (FIG. 2), to determine air flow of the mouth and/or nose of the user, to determine a time when the user enters the bed (FIG. 2), and to determine a time when the user exits the bed. In some implementations, the camera 232 includes a wide angle lens or a fish eye lens.

The infrared (IR) sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120 and/or the associated headgear (e.g., straps, etc.).

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 174 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 174 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The Light Detection and Ranging (LiDAR) sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 100, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 132 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 (FIG. 1) includes a display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 100 also includes an activity tracker 270. The activity tracker 270 is generally used to aid in generating physiological data associated with the user. The activity tracker 270 can include one or more of the sensors 210 described herein, such as, for example, the motion sensor 138 (e.g., one or more accelerometers and/or gyroscopes), the PPG sensor 154, and/or the ECG sensor 156. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 100 also includes a blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra wideband ranges (which may include spreading, PRN codes or impulse systems).

While the control system 200 and the memory device 204 are described and shown in FIG. 1 as being a separate and distinct component of the system 100, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user 20 has laid or sat down in the bed 40 (or another area or object on which they intend to sleep), and has turned on the respiratory therapy device 110 and donned the user interface 120. The sleep session can thus include time periods (i) when the user 20 is using the respiratory therapy system 100, but before the user 20 attempts to fall asleep (for example when the user 20 lays in the bed 40 reading a book); (ii) when the user 20 begins trying to fall asleep but is still awake; (iii) when the user 20 is in a light sleep (also referred to as stage 1 and stage 2 of non-rapid eye movement (NREM) sleep); (iv) when the user 20 is in a deep sleep (also referred to as slow-wave sleep, SWS, or stage 3 of NREM sleep); (v) when the user 20 is in rapid eye movement (REM) sleep; (vi) when the user 20 is periodically awake between light sleep, deep sleep, or REM sleep; or (vii) when the user 20 wakes up and does not fall back asleep.

The sleep session is generally defined as ending once the user 20 removes the user interface 120, turns off the respiratory therapy device 110, and gets out of bed 40. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user 20, ending when the respiratory therapy device 110 stops supplying the pressurized air to the airway of the user 20, and including some or all of the time points in between, when the user 20 is asleep or awake.

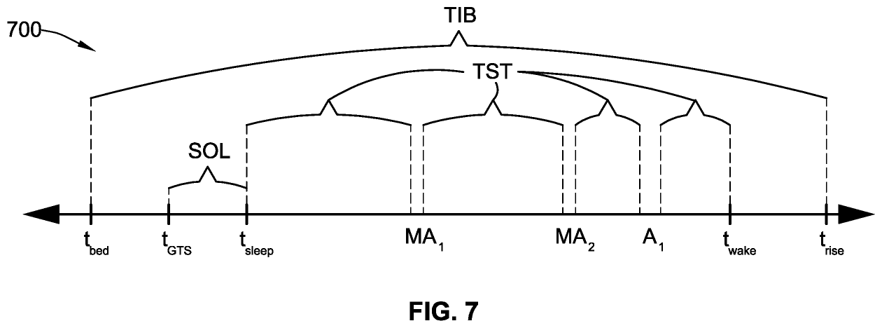

Referring to the timeline 700 in FIG. 7 the enter bed time t_{bed} is associated with the time that the user initially enters the bed (e.g., bed 40 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least bed 15 minutes, at least 20 minutes, at least 30 minutes, at least 1 hour, etc.). The enter bed time time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 700 of FIG. 7, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

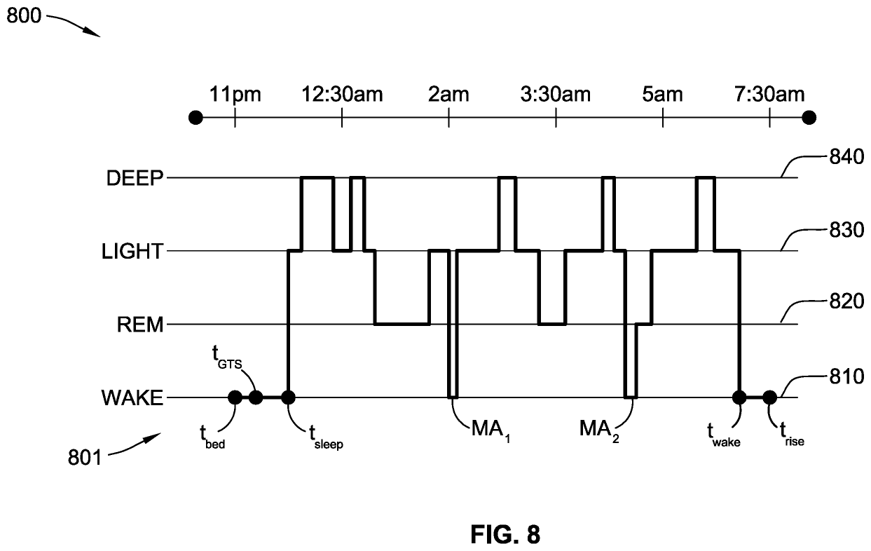

Referring to FIG. 8, an exemplary hypnogram 800 corresponding to the timeline 700 (FIG. 7), according to some implementations, is illustrated. As shown, the hypnogram 800 includes a sleep-wake signal 801, a wakefulness stage axis 810, a REM stage axis 820, a light sleep stage axis 830, and a deep sleep stage axis 840. The intersection between the sleep-wake signal 801 and one of the axes 810-840 is indicative of the sleep stage at any given time during the sleep session.

The sleep-wake signal 801 can be generated based on physiological data associated with the user (e.g., generated by one or more of the sensors 210 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 800 is shown in FIG. 8 as including the light sleep stage axis 830 and the deep sleep stage axis 840, in some implementations, the hypnogram 800 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 204.

The hypnogram 800 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 7), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA 2 shown in FIG. 7), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time a the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 218, the microphone 220, the camera 232, or any combination thereof. The go-to-sleep time can be determined based on, for example, data from the motion sensor 218 (e.g., data indicative of no movement by the user), data from the camera 232 (e.g., data indicative of no movement by the user and/or that the user has turned off the lights) data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

Referring to FIGS. 9A-9C, illustrated are steps for determining whether the fit of a user interface is proper. Specifically referring to FIG. 9A, a user 900 has a user interface 902 donned on his/her face 901. The user interface 902 can be any user interface discussed above that includes the cushion 902. In the specific steps illustrated in FIGS. 9A-9C, which concern a visual inspection, the cushion includes an indicator (e.g., indicator 434). The user 900 initially has the user interface 902 on the face 901. According to some implementations, the user interface 902 may be donned on the face 901 for any period of time. Alternatively, the user interface 902 may be donned on the face 901 for a period of time that allows the user interface 902 to fully settle on the face 901. For example, the face 901 naturally has some elasticity. Allowing the user interface 902 to be donned on the face 901 for a period of time before removal allows for the elasticity of the face 901 to reach a steady state so that an accurate representation of the seal between the face 901 and the user interface 902 is obtained.

Referring to FIG. 9B, in the case of a dye as the indicator on the user interface, after removing the user interface 902, a dye outline 906 is formed on the face 901 resulting from a transfer of dye (e.g., indicator 434) from the user interface to the face 901. A consistent, solid dye outline 906 indicates a proper fit of the user interface 902 on the face 901 of the user 900. Alternatively, and as shown in FIG. 9B, a discontinuity 908, such as the illustrated gap, in the dye outline 906 indicates an improper fit of the user interface 902 and the face 901. More specifically, the discontinuity 908 indicates an area where pressurized air may escape from between the face 901 and the user interface 902 during therapy.

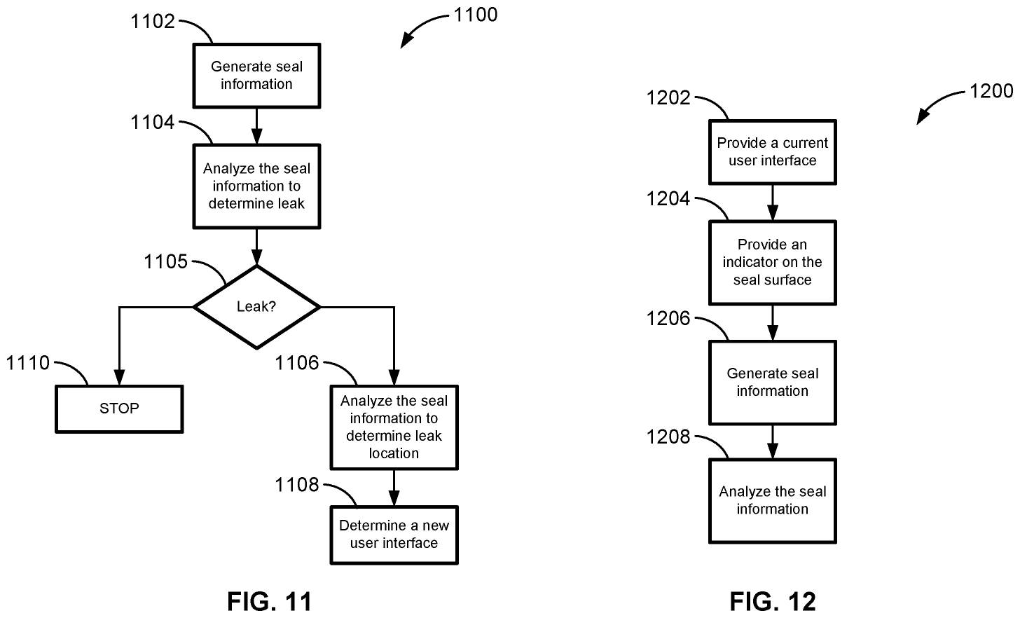

According to some embodiments, depending on the severity of the improper fit of the user interface 902, there may be more than one discontinuity 908, such as two, three, four, five, etc. gaps 908. In some implementations, the discontinuity 908 of the dye outline 906 may appear instead as a lighter shade of the dye, a patchier region of the dye, and the like, besides an absolute gap. However, in whatever way the discontinuity 908 of the dye outline 906 manifests, the outline dye 906 can be visually scanned by a camera, such as by a camera in a smart device, for determining whether the fit of the user interface 902 is proper, as discussed further below with respect to FIGS. 11 and 12. If the user interface 902 is determined to not fit properly, the user may obtain a new user interface. The new user interface may even be recommended based on the specific details of the discontinuity, such as the severity and the location. Further, the location and type of discontinuity in the outline 906 can be used for determining a new user interface for the user to try based on specific user interfaces being associated with correcting specific issues in seals between the user interface and the face.

Referring to FIG. 9C, the cushion 930 of the user interface 902 in FIG. 9A can instead have an indicator 934, such as being on the seal surface 932, as discussed above with respect to FIGS. 4C and 4D. Like the discontinuity 908 in the dye outline 906 in FIG. 9B, a discontinuity 936 in the indicator 934 upon removing the cushion 930 from the face 901 of the user 900 (FIG. 9A) similarly indicates the quality of the fit of the user interface 900 against the face 901 of the user 900.

Referring to FIG. 10, illustrated is a step in determining the fit of a user interface based on audio, according to some implementations of the present disclosure. A user 1000 is wearing a user interface 1002 on the face 1001, and the user interface 1002 that includes a cushion 1004. The cushion 1004 does not require an indicator. Instead, the cushion 1004 can be any cushion described herein, including those that do and do not have an indicator. The user interface 1002 is connected to a respiratory therapy device, such as the respiratory therapy device 110. The respiratory therapy device provides positive airway pressure to the user 1000. As the positive airway pressure is provided to the cushion 1004, discontinuities in the seal region between the cushion 1004 and the face 1001 of the user 1000 based on the user interface 1002 not having a proper fit results in pressurized air escaping from the cushion 1004, as represented by the dashed lines 1006. The escaping pressurized air 1006 also makes a noise, such as a slight hissing. A listening device 1008 can be presented at the user interface 1002 to listen for the associated hissing sound of the escaping pressurized air 1006. Moreover, the listening device 1008 can be moved around the user interface 1002 to trace the seal region for localizing a location of the sound associated with the escaping pressurized air 1006. Based on, for example, the presence, the location, and/or the amplitude of the escaping pressurized air 1006, and its associated sound, a determination can be made regarding whether the fit between the user interface 1002 and the face 1001 is proper.

According to some implementations, the listening device 1008 can be any device that is able to convert sound into a signal for subsequent processing. For example, the listening device 1008 can be any microphone. According to some implementations, the listening device 1008 can be a microphone within a wearable device or a smart phone. Such a wearable device could be, for example, a pair of over-the-ear headphones, earbuds, hearing aids, etc., either with a microphone or with one or more speakers configured as a microphone. Such a wearable device also could be a smart watch with a microphone, or any other wearable device that has a microphone.

According to some implementations, the listening device 1008 can be connected to (wired or wirelessly) or integrated in a smart phone. The smart phone can execute an application, such as a patient engagement application. The application can process information from the listening device 1008 for determining a likelihood of a leak and a likely location of the leak.

Alternatively, the listening device 1008 can be connected to (wired or wirelessly) or integrated in a respiratory therapy device (e.g., respiratory therapy device 110). For example, one or more listening devices 1008 can be integrated into the user interface or in the end of a user interface conduit (e.g., user interface conduit 590) that connects to the user interface.

The respiratory therapy device can perform the subsequent processing to determine a likelihood of leak and a likely location of the leak. The respiratory therapy device can then present on its graphical user interface the likelihood of the leak and the likely location of the leak. Alternatively, the respiratory therapy device can be connected (wired or wirelessly) to the smart phone. After the smart phone performs the required processing, the information on the likelihood of the leak and the likely location of the leak the can be sent to the respiratory therapy device for presentation as above on its graphical user interface.

According to some implementations, a single listening device 1008 may be used, and the user can move the single listening device 1008 around the perimeter of the user interface. Alternatively, multiple listening devices in fixed locations can be used to eliminate the need for moving the listening device 1008. The information acquired by the multiple listening devices could be processed to triangulate location(s) of leak. For example, multiple listening devices 1008 in the form of wireless earbuds (e.g., Apple® AirPods®) can be worn in the ear (i.e., fixed locations).

One advantage of having multiple listening devices 1008 is that leak detection can be monitored without any user interaction, such as moving the listening device. Therefore, there are more situations in which the listening devices 1008 can be listening for a leak, such as while the user sleeps.

According to some implementations, based on the processed signal from the listening device 1008, the user may be instructed to adjust the user interface accordingly, such as tightening or loosening a specific headgear strap (e.g., tighten top right headgear strap). After the adjustment, the listening device 1008 can again listen for a leak to determine if the adjustment correct the leak or if perhaps a new user interface is needed.

Referring to FIG. 11, a method 1100 for determining whether a user interface fits properly, according to some implementations of the present disclosure, is illustrated. One or more steps of the method 1100 can be implemented using any element or aspect of the system 100 described herein.

At step 1102, seal information associated with a seal region between a face of a user and a current user interface donned on the face of the user is generated. The seal information can be generated as a result of various different implementations described below.

According to some implementations, at least one microphone is used to scan the seal region between the face of the user and the current user interface donned on the face of the user while positive airway pressure is being supplied to the user through the current user interface. The scanning can include bringing the microphone near the current user interface so that the microphone can detect sound generated by escaping pressurized air. For example, the seal information can be based on audio information generated from nasal resistance that is detected by the at least one microphone located near the current user interface. The scanning can also include tracing the seal region with the at least one microphone during the scanning of the seal region such that the seal information is generated as a function of a position along the seal region. The location of the at least one microphone can be determined relative to the user interface based on information acquired from one or more gyroscopes, one or more accelerometers, or a combination thereof. The tracing can begin at a set position relative to the user interface to relate the position of the at least one microphone to the user interface. Alternatively, one or more sensors in the user interface can coordinate with one or more sensors in the device with the at least one microphone to relate the position of the at least one microphone to the user interface.

According to some implementations, at least one camera is used to scan the seal region between the face of the user and the current user interface donned on the face of the user. The scanning can involve photographs, videos, or a combination thereof. The photographs and videos can be two-dimensional, three-dimensional, or a combination thereof. Scanning the seal region can include scanning the seal region relative to the face of the user, such as described with respect to FIG. 9B above. Alternatively, or in combination, scanning the seal region can include scanning the seal region relative to the user interface, such as described above with respect to FIG. 9C above. As a result, the seal information can be generated by the at least one camera during the scanning of the seal region. For example, the scanning of the seal region can occur after removing the current user interface from being donned on the face. The generated seal information can then be based on visually detecting one or more indentations on the face of the user or on the current user interface along the seal region. Like the at least one microphone discussed above, the at least one camera can be on any device that includes a camera and the ability to process the generated information and/or transmit the generated information for remote processing.

When a camera is used to generate the seal information, an indicator, such as a dye, can be placed on a surface of the current user interface that makes contact with the face of the user when the current user interface is donned on the face of the user. The dye can be configured to transfer to the face of the user. Alternatively, the dye can be configured to remain on the user interface. Alternatively, the dye can be configured to transfer partially to the face of the user and configured to remain partially on the user interface. The scanning of the seal region can then include scanning the dye left on the face of the user around the seal region after removing the current user interface from being donned on the face. The scanning of the seal region can then include scanning the dye left remaining on the face of the user around the seal region after removing the current user interface from being donned on the face. The seal information is then generated by the at least one camera during the scanning of the dye.

In some implementations, when at least one camera is used to scan the seal region, prior to this step, the at least one camera can be used to scan the face of the user prior the current user interface being donned on the face of the user to generate face information. For example, the face information can provide details regarding the structure or surface of the face that relate to or can be associated with discontinuities between the face and the user interface. The details can be used with respect to the seal information in the subsequent steps below. The seal information can then be generated based, at least in part, on the face information.

At step 1104, the seal information is analyzed to determine whether a leak exists in the seal region. The method disclosed above that is used to generate the seal information determines how the seal information is analyzed. Thus, the analysis can be based on various visual and/or audio analysis methods. In the case of audio, and specifically nasal resistance, the detected nasal resistance can be compared to a baseline nasal resistance. The comparison determines whether there is a leak. In the case of audio, and specifically detecting a leak of pressurized air, the detected sound associated with the leak of pressurized air indicates a leak within the seal region. In the case of an indicator, such as a dye or any other indicator disclosed above, a discontinuity indicates a leak within the seal region, as described above. If after step 1104 it is determined that a leak does not exist, the method 1100 stops at step 1110. However, if a leak is detected, the method can proceed to step 1106.

At step 1106, and if a leak exists, the seal information is analyzed to determine a location of the leak within the seal region. The method disclosed above that is used to generate the seal information determines how the seal information is analyzed. Thus, the analysis can be based on various visual and/or audio analysis methods. The location of the leak can be determined based on the location of the noise associated with the leak. The location of the leak can be determined based on the location of the discontinuity. The location of the leak can also be based on the face information, if generated in step 1102, for further pinpointing the location of the leak in the seal region based on the details of the face of the user.

According to some implementations, the face information may also be used to identify users who might be suitable for alternative therapies, such as positional OSA treatment, use of a mandibular device, etc.

After step 1106, at step 1108, a new user interface to replace the current user interface is determined based on the current user interface and the location of the leak. The new user interface can be selected based on a known relationship between a specific leak location and a user interface that is associated with correcting or preventing a leak in the specific leak location. The new user interface can be a completely different type of user interface than the current user interface. For example, the cushion of the current user interface may cover the nose and the mouth of the user. If the leak is associated with a location around the mouth, the new user interface can be selected so as to cover only the nose, or vice versa. Once the user receives the new user interface, the method 1100 can be repeated again to determine if the new user interface, now considered the current user interface, provides a better seal with the face of the user.

According to some implementations, the method 1100 can be performed with the current user interface donned on the face of the user according to a comfortable configuration. Alternatively, the method 1100 can be performed initially with the user interface donned on the user in an over-tightened configuration. For example, the headgear used to attach the user interface to the user may be overtightened. The method 1100 can be performed one or more times, with each time the method being performed the user loosening the headgear. When a leak is first detected, the tightness of the headgear can be taken into consideration when determining whether the fit of the user interface is proper. For example, if the tightness of the headgear is looser than a recommended setting, but there was no leak when the tightness was at a recommended setting, the user interface may still be proper.

According to some implementations, the user can be asked for input on the fit of the user interface. Such input can include, for example, whether the user believes the fit is proper, whether the fit is comfortable, whether the user hears a sound related to a leak with the user interface, and the like. The user's input can be used in the analysis of whether there is a leak, whether the fit of the user interface is proper, or both. For example, the user's input regarding a sound related to a leak can be used to confirm a suspected presence of a leak based on audio information.

Referring to FIG. 12, a method 1200 for determining whether a user interface fits properly, according to some alternative implementations of the present disclosure, is illustrated. One or more steps of the method 1200 can be implemented using any element or aspect of the system 100 described herein.

At step 1202, a current user interface connected to a respiratory therapy system is provided. The current user interface includes a seal surface where the current user interface contacts a face of a user with the current user interface donned on the face of the user.

At step 1204, an indicator is provided on the seal surface of the user interface. The indicator is configured to contact the face of the user when the current user interface is donned on the face of the user. According to some implementations, the indicator is a contour-forming material that develops an impression of topology of the face of the user. According to some implementations, the indicator is a dye. The dye can be moisture-released from the peelable layer so as to transfer to the face of the user when the current user interface is donned on the face of the user. The dye can be configured to transfer to the face of the user when the current user interface is donned on the face of the user. Alternatively, the dye can be configured to remain on the seal surface of the current user interface upon the current user interface being removed from the face of the user. Alternatively, the indicator can be any indicator disclosed herein.

At step 1206, seal information associated with a seal region between the face of the user and the seal surface of the current user interface is generated based on the indicator and upon the current user interface being removed from the face of the user. Similar to step 1102 for the method 1100, the seal information can be generated based on various methods discussed above with respect to using an indicator, where a discontinuity, or a discontinuity and severity, of the indicator, indicates a leak within the seal region.

At step 1208, the seal information is analyzed to determine whether the current user interface fits properly. The analysis can occur similar to steps 1104 and 1106 discussed above, focusing on the implementations with an indicator. If the current user interface is determined to fit properly, the user can continue using the current user interface. If the current user interface includes a peelable layer, the peelable layer can be removed from the seal surface for continued use of the current user interface. If the current user interface is determined to not fit properly, the current user interface can be returned for a new user interface. Further, a new user interface can be recommended based on the specific deficiency related to the current user interface. The method of 1200 can repeat with the new user interface now considered the current user interface.

### Alternative Implementation Section

Implementation 1. A method comprising: generating seal information associated with a seal region between a face of a user and a current user interface donned on the face of the user; analyzing the seal information to determine whether a leak exists in the seal region; and if the leak exists: analyzing the seal information to determine a location of the leak within the seal region; and determining a new user interface to replace the current user interface based on the current user interface and the location of the leak.

Implementation 2. The method of implementation 1, further comprising: scanning, with at least one microphone, the seal region between the face of the user and the current user interface donned on the face of the user while positive airway pressure is being supplied to the user through the current user interface, wherein the seal information is generated by the at least one microphone during the of scanning the seal region.

Implementation 3. The method of implementation 2, wherein the microphone is within a wearable device or a smart phone.

Implementation 4. The method of implementation 2 or implementation 3, further comprising: tracing the seal region with the at least one microphone during the scanning of the seal region such that the seal information is generated as a function of a position along the seal region.

Implementation 5. The method of any one of implementations 1 to 4, further comprising: scanning, with at least one camera, the seal region between the face of the user and the current user interface donned on the face of the user, wherein the seal information is generated by the at least one camera during the scanning of the seal region.

Implementation 6. The method of implementation 5, further comprising: scanning, with the at least one camera, the face of the user prior the current user interface being donned on the face of the user to generate face information, wherein the seal information is generated based, at least in part, on the face information.

Implementation 7. The method of implementation 5 or implementation 6, further comprising: placing a dye on a surface of the current user interface that makes contact with the face of the user when the current user interface is donned on the face of the user, wherein the scanning of the seal region includes scanning the dye left on the face of the user around the seal region after removing the current user interface from being donned on the face, and the seal information is generated by the at least one camera during the scanning of the dye.

Implementation 8. The method of any one of implementations 5 to 7, wherein the scanning of the seal region occurs after removing the current user interface from being donned on the face, and the generated seal information is based on visually detecting one or more indentations on the face of the user or on the current user interface along the seal region.

Implementation 9. The method of any one of implementations 1 to 8, wherein the generating the seal information occurs after a period of time has elapsed from when the current user interface was donned on the face of the user so that the current user interface is fully settled on the face of the user.

Implementation 10. The method of any one of implementations 1 to 9, wherein the current user interface includes a dye that is time-activated, moisture activated, photochromic, ultraviolet light sensitive, or a combination thereof.

Implementation 11. The method of implementation 10, wherein the dye is on a peelable layer on the current user interface.

Implementation 12. The method of any one of implementations 1 toll, wherein the seal information is based on audio information generated from nasal resistance.

Implementation 13. A method comprising: providing a current user interface connected to a respiratory therapy system, the current user interface including a seal surface where the current user interface contacts a face of a user with the current user interface donned on the face of the user; providing an indicator on the seal surface of the user interface, the indicator being configured to contact the face of the user when the current user interface is donned on the face of the user; generating seal information associated with a seal region between the face of the user and the seal surface of the current user interface based on the indicator and upon the current user interface being removed from the face of the user; and analyzing the seal information to determine whether the current user interface fits properly.

Implementation 14. The method of implementation 13, further comprising: continuing to use the current user interface if the current user interface is determined to fit properly; and returning the current user interface for a new user interface if the current user interface is determined to not fit properly.

Implementation 15. The method of implementation 13 or implementation 14, wherein the indicator is a contour-forming material that develops an impression of topology of the face of the user.

Implementation 16. The method of any one of implementations 13 to 15, wherein the indicator is a dye.

Implementation 17. The method of implementation 16, wherein the dye is configured to transfer to the face of the user when the current user interface is donned on the face of the user.

Implementation 18. The method of implementation 16 or implementation 17, wherein the dye is configured to remain on the seal surface of the current user interface upon the current user interface being removed from the face of the user.

Implementation 19. The method of any one of implementations 16 to 18, wherein the dye is time-activated, moisture activated, photochromic, ultraviolet light sensitive, or a combination thereof.

Implementation 20. The method of any one of implementations 16 to 19, providing the dye on or within a peelable layer on the seal surface of the user interface.

Implementation 21. The method of implementation 20, wherein the dye is moisture-released from the peelable layer so as to transfer to the face of the user when the current user interface is donned on the face of the user.

Implementation 22. The method of implementation 20 or implementation 21, further comprising: removing the peelable layer from the seal surface for continued use of the current user interface if the current user interface is determined to fit properly.

Implementation 23. A system comprising: a control system comprising one or more processors; and a memory having stored thereon machine readable instructions; wherein the control system is coupled to the memory, and the method of any one of implementations 1 to 22 is implemented when the machine executable instructions in the memory are executed by at least one of the one or more processors of the control system.

Implementation 24. A system for communicating one or more indications to a user, the system comprising a control system configured to implement the method of any one of implementations 1 to 22.

Implementation 25. A computer program product comprising instructions which, when executed by a computer, cause the computer to carry out the method of any one of implementations 1 to 22.

Implementation 26. The computer program product of implementation 25, wherein the computer program product is a non-transitory computer readable medium.

Implementation 27. A user interface comprising: a frame and headgear that position the user interface on a face of a user relative to an airway of the user, with the user interface donned on the face of the user; a cushion that is supported against the face of the user by the frame and the headgear to define a seal region around the airway of the user, with the user interface donned on the face of the user, the cushion including a seal surface where the cushion contacts the face of the user at the seal region; and an indicator on the seal surface, the indicator being configured to contact the face of the user when the user interface is donned on the face of the user for determining whether the user interface fits properly.

Implementation 28. The user interface of implementation 27, further comprising: a peelable layer on the seal surface, wherein the indicator is the peelable layer, is on the peelable layer, is in the peelable layer, or a combination thereof.

Implementation 29. The user interface of implementation 27 or implementation 28, wherein the indicator is a contour-forming material that develops an impression of topology of the face of the user, and the impression of topology can be analyzed for determining whether the user interface fits properly.

Implementation 30. The user interface of any one of implementations 27 to 29, wherein the indicator is a dye that makes contact with the face of the user when the user interface is donned on the face of the user.

Implementation 31. The user interface of implementation 30, wherein the dye is configured to transfer to the face of the user when the user interface is donned on the face of the user, and the transferred dye on the face of the user can be visually scanned for determining whether the user interface fits properly.

Implementation 32. The user interface of implementation 30 or implementation 31, wherein the dye is configured to activate when in contact with the face of the user but remain on the seal surface of the user interface upon the user interface being removed from the face of the user, and activated portions of the dye on the seal surface can be visually scanned for determining whether the user interface fits properly.

Implementation 33. The user interface of any one of implementations 30 to 32, wherein the dye is time-activated, moisture activated, photochromic, ultraviolet light sensitive, or a combination thereof.

Implementation 34. The user interface of any one of implementations 30 to 33, further comprising: a peelable layer on the seal surface, wherein the dye is on the peelable layer, is in the peelable layer, or a combination thereof.

Implementation 35. The user interface of implementation 34, wherein the dye is moisture-released from the peelable layer so as to transfer to the face of the user when the current user interface is donned on the face of the user.

Implementation 36. A cushion of a user interface, the cushion comprising: a seal surface that contacts a face of a user when the user interface is donned on the face of the user; and an indicator on the seal surface, the indicator being configured to contact the face of the user when the user interface is donned on the face of the user for determining whether the user interface fits properly.

Implementation 37. The cushion of implementation 36, further comprising: a peelable layer on the seal surface, wherein the indicator is the peelable layer, is on the peelable layer, is in the peelable layer, or a combination thereof.

Implementation 38. The cushion of implementation 36 or implementation 37, wherein the indicator is a contour-forming material that develops an impression of topology of the face of the user, and the impression of topology can be analyzed for determining whether the user interface fits properly.

Implementation 39. The cushion of any one of implementations 36 to 38, wherein the indicator is a dye that makes contact with the face of the user when the user interface is donned on the face of the user.

Implementation 40. The cushion of implementation 39, wherein the dye is configured to transfer to the face of the user when the user interface is donned on the face of the user, and the transferred dye on the face of the user can be visually scanned for determining whether the user interface fits properly.

Implementation 41. The cushion of implementation 39 or implementation 40, wherein the dye is configured to activate when in contact with the face of the user but remain on the seal surface of the user interface upon the user interface being removed from the face of the user, and activated portions of the dye on the seal surface can be visually scanned for determining whether the user interface fits properly.

Implementation 42. The cushion of any one of implementations 39 to 41, wherein the dye is time-activated, moisture activated, photochromic, ultraviolet light sensitive, or a combination thereof.

Implementation 43. The cushion of any one of implementations 39 to 42, further comprising: a peelable layer on the seal surface, wherein the dye is on the peelable layer, is in the peelable layer, or a combination thereof.

Implementation 44. The cushion of implementation 43, wherein the dye is moisture-released from the peelable layer so as to transfer to the face of the user when the current user interface is donned on the face of the user.

One or more elements or aspects or steps, or any portion(s) thereof, from one or more of any of the above implementations above can be combined with one or more elements or aspects or steps, or any portion(s) thereof, from one or more of any of the other above implementations or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

What is claimed is:

1. A method comprising:
generating seal information associated with a seal region between a face of a user and a current user interface donned on the face of the user;
analyzing the seal information to determine whether a leak exists in the seal region; and
if the leak exists:
analyzing the seal information to determine a location of the leak within the seal region; and
determining a new user interface to replace the current user interface based on the current user interface and the location of the leak.

2. The method of claim 1, further comprising:
scanning, with at least one microphone, the seal region between the face of the user and the current user interface donned on the face of the user while positive airway pressure is being supplied to the user through the current user interface,
wherein the seal information is generated by the at least one microphone during the of scanning the seal region.

3. The method of claim 2, further comprising:
tracing the seal region with the at least one microphone during the scanning of the seal region such that the seal information is generated as a function of a position along the seal region.

4. The method of claim 1, further comprising:
scanning, with at least one camera, the seal region between the face of the user and the current user interface donned on the face of the user,
wherein the seal information is generated by the at least one camera during the scanning of the seal region.

5. The method of claim 4, further comprising:
placing a dye on a surface of the current user interface that makes contact with the face of the user when the current user interface is donned on the face of the user,
wherein the scanning of the seal region includes scanning the dye left on the face of the user around the seal region after removing the current user interface from being donned on the face, and the seal information is generated by the at least one camera during the scanning of the dye.

6. The method of claim 1, wherein the generating the seal information occurs after a period of time has elapsed from when the current user interface was donned on the face of the user so that the current user interface is fully settled on the face of the user.

7. The method of claim 1, wherein the current user interface includes a dye that is time-activated, moisture activated, photochromic, ultraviolet light sensitive, or a combination thereof.

8. A method comprising:
providing a current user interface connected to a respiratory therapy system, the current user interface including a seal surface where the current user interface contacts a face of a user with the current user interface donned on the face of the user;
providing an indicator on the seal surface of the user interface, the indicator being configured to contact the face of the user when the current user interface is donned on the face of the user;
generating seal information associated with a seal region between the face of the user and the seal surface of the current user interface based on the indicator and upon the current user interface being removed from the face of the user; and
analyzing the seal information to determine whether the current user interface fits properly.

9. The method of claim 8, further comprising:
continuing to use the current user interface if the current user interface is determined to fit properly; and
returning the current user interface for a new user interface if the current user interface is determined to not fit properly.

10. The method of claim 8, wherein the indicator is a dye.

11. The method of claim 10, wherein the dye is configured to transfer to the face of the user when the current user interface is donned on the face of the user.

12. The method of claim 10, wherein the dye is configured to remain on the seal surface of the current user interface upon the current user interface being removed from the face of the user.

13. The method of claim 10, wherein the dye is time-activated, moisture activated, photochromic, ultraviolet light sensitive, or a combination thereof.

14. The method of claim 10, providing the dye on or within a peelable layer on the seal surface of the user interface.

15. A user interface comprising:
a frame and headgear that position the user interface on a face of a user relative to an airway of the user, with the user interface donned on the face of the user;
a cushion that is supported against the face of the user by the frame and the headgear to define a seal region around the airway of the user, with the user interface donned on the face of the user, the cushion including a seal surface where the cushion contacts the face of the user at the seal region; and
an indicator on the seal surface, the indicator being configured to contact the face of the user when the user interface is donned on the face of the user for determining whether the user interface fits properly.

16. The user interface of claim 15, further comprising:
a peelable layer on the seal surface, wherein the indicator is the peelable layer, is on the peelable layer, is in the peelable layer, or a combination thereof.

17. The user interface of claim 15, wherein the indicator is a contour-forming material that develops an impression of topology of the face of the user, and the impression of topology can be analyzed for determining whether the user interface fits properly.

18. The user interface of claim 15, wherein the indicator is a dye that makes contact with the face of the user when the user interface is donned on the face of the user.

19. The user interface of claim 18, wherein the dye is time-activated, moisture activated, photochromic, ultraviolet light sensitive, or a combination thereof.

20. The user interface of claim 18, further comprising:
a peelable layer on the seal surface, wherein the dye is on the peelable layer, is in the peelable layer, or a combination thereof.
